SEQUENCE LISTING (4 sequences)

SEQ ID NO: 1 - DNA, 1143 bp, Artificial Sequence
  Description of Artificial Sequence Synthetic nucleotide sequence
atggtcgaatacacaaacacattcaaagttgctgcggtgcaggcacagcctgtgtggttcgacgcggccaaaacggtcgacaagaccgtgtccatcatcgcggaagcagcccggaacgggtgcgagctcgttgcgtttcccgaggtattcatcccggggtacccgtaccacatctgggtcgacagcccgctcgccggaatggcgaagttcgccgtgcgctaccacgagaattccctgacgatggacagcccgcacgtacagcggttgctcgatgccgcccgcgaccacaacatcgccgtagtggtgggaatcagcgagcgggatggcggcagcttgtacatgacccagctcatcatcgacgccgatgggcaactggtcgcccgacgccgcaagctcaagcccacccacgtcgagcgttcggtatacggagaaggaaacggctcggatatctccgtgtacgacatgcctttcgcacggcttggcgcgctcaactgctgggagcatttccagacgctcaccaagtacgcaatgtactcgatgcacgagcaggtgcacgtcgcgagctggcctggcatgtcgctgtaccagccggaggtccccgcattcggtgtcgatgcccagctcacggccacgcgtatgtacgcactcgagggacaaaccttcgtggtctgcaccacccaggtggtcacaccggaggcccacgagttcttctgcgagaacgaggaacagcgaaagttgatcggccgaggcggaggtttcgcgcgcatcatcgggcccgacggccgcgatctcgcaactcctctcgccgaagatgaggaggggatcctctacgccgacatcgatctgtctgcgatcaccttggcgaagcaggccgctgaccccgtgggccactactcacggccggatgtgctgtcgctgaacttcaaccagcgccgcaccacgcccgtcaacaccccactttccaccatccatgccacgcacacgttcgtgccgcagttcggggcactcgacggcgtccgtgagctcaacggagcggacgaacagcgcgcattgccctccacacattccgacgagacggaccgggcgacagcaccctctgactcgggcgcacccgtggcgcctccgaagcgccacggtgtgtga

SEQ ID NO: 2 - protein, 380 aa, Artificial Sequence
  Description of Artificial Sequence Synthetic peptide sequence
MVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHNIAVVVGISERDGGSLYMTQLIIDADGQLVARRRKLKPTHVERSVYGEGNGSDISVYDMPFARLGALNCWEHFQTLTKYAMYSMHEQVHVASWPGMSLYQPEVPAFGVDAQLTATRMYALEGQTFVVCTTQVVTPEAHEFFCENEEQRKLIGRGGGFARIIGPDGRDLATPLAEDEEGILYADIDLSAITLAKQAADPVGHYSRPDVLSLNFNQRRTTPVNTPLSTIHATHTFVPQFGALDGVRELNGADEQRALPSTHSDETDRATAPSDSGAPVAPPKRHGV

SEQ ID NO: 3 - DNA, 1101 bp, Artificial Sequence
  Description of Artificial Sequence Synthetic nucleotide sequence
atggtcgaatacacaaacacattcaaagttgctgcggtgcaggcacagcctgtgtggttcgacgcggccaaaacggtcgacaagaccgtgtccatcatcgcggaagcagcccggaacgggtgcgagctcgttgcgtttcccgaggtattcatcccggggtacccgtaccacatctgggtcgacagcccgctcgccggaatggcgaagttcgccgtgcgctaccacgagaattccctgacgatggatagcccgcacgtacagcggttgctcgatgccgcccgcgaccacagcatcgccgtagtggtgggaatcagcgagcgggatggcggcagcttgtacatgacccagctcatcatcgacgccgatgggcagctggtcgcccgacgccgcaagctcaagcccacccacgtcgagcgttcggtatacggagaaggaaacggctcggatatctccgtgtacgacatgcctttcgcgcggctcggcgcgctcaactgctgggagcatttccagacgctcaccaagtacgcaatgtactcgatgcacgagcaggtgcacgtcgcgagctggcctggcatgtcgctgtaccagccggaggtccccgccttcggtgtcgatgcccagctcacggccacgcgtatgtatgcactcgagggacaaaccttcgtggtttgcaccacccaggtggtcacgccggaggcccacgagttcttctgcgagaacgaggaacagcgaaagctgatcggccgaggcggaggtttcgcgcggatcatcgggcccgacggccgcgatctcgcaactcctctcgccgaagatgaggaggggatcctctacgccgacatcgatctgtctgcgatcaccttggcgaagcaggccgccgaccccgtaggccactactcacggccggatgtgctgtcgctgaacttcaaccagcgccgcaccacgcccgtcaacaccccactttccaccatccatgccacgcacacgttcgtgccgcagttcggggcactcgacggcgtccgtgagctcaacggagcggacgaacagcgcgcattgccctccacacattccgacgagacggaccgggcgacagcctccatctga

SEQ ID NO: 4 - protein, 366 aa, Artificial Sequence
  Description of Artificial Sequence Synthetic peptide sequence
MVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHSIAVVVGISERDGGSLYMTQLIIDADGQLVARRRKLKPTHVERSVYGEGNGSDISVYDMPFARLGALNCWEHFQTLTKYAMYSMHEQVHVASWPGMSLYQPEVPAFGVDAQLTATRMYALEGQTFVVCTTQVVTPEAHEFFCENEEQRKLIGRGGGFARIIGPDGRDLATPLAEDEEGILYADIDLSAITLAKQAADPVGHYSRPDVLSLNFNQRRTTPVNTPLSTIHATHTFVPQFGALDGVRELNGADEQRALPSTHSDETDRATASI

(19) United States
(12) Patent Application Publication
(10) Pub. No.: US 2002/0137153 A1
(43) Pub. Date: Sep. 26, 2002

(54) Enantioselective production of amino carboxylic acids

(76) Inventors: Sandra W. Ramer, Sunnyvale, CA (US); Gjalt Huisman, San Carlos, CA (US); Jim Millis, Kohler, WI (US); Roger Sheldon, Rijswijk (NL); Stephen delCardayre, Belmont, CA (US); Matthew Tobin, San Jose, CA (US); Anthony Cox, Mountain View, CA (US); S. Christopher Davis, San Francisco, CA (US)

Correspondence Address: LAW OFFICES OF JONATHAN ALAN QUINE, P O BOX 458, ALAMEDA, CA, 94501

(21) Appl. No.: 09/972,186

(22) Filed: Oct. 4, 2001

Related U.S. Application Data
(60) Provisional application No. 60/238,563, filed on Oct. 4, 2000.

(51) Int. Cl. C12P 13/02; C12N 9/78

(52) U.S. Cl. 435/129; 435/227

(57) ABSTRACT

Enantioselective or enantiospecific nitrilases and nitrile hydratases are used to produce R or S enantiomers of amides, and carboxylic acids. R-amino acids and S-amino acids are produced using such enantioselective enzymes. In addition, methods of producing and screening enantioselective nitrilases and nitrile hydratases are provided.

### CROSS REFERENCE TO RELATED APPLICATIONS

Pursuant to 35 U.S.C. §119(e) and any other applicable statute or rule, the present application claims benefit of and priority to U.S. patent application Ser. No. 60/238,563, filed Oct. 4, 2000, entitled “Enantioselective Production of Amino Carboxylic Acids,” the disclosure of which is incorporated herein by reference in their entirety for all purposes.

### COPYRIGHT NOTIFICATION

Pursuant to 37 C.F.R. 1.71(c), a portion of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Amino acids, amides, and carboxylic acids often comprise a chiral center that leads to the compound existing in two different enantiomers, an R-enantiomer and an S-enantiomer. In many applications, it is desirable to use only one of the possible enantiomers, yet many of the methods known in the art for producing such compounds yield a racemic mixture of both enantiomers. For example, Strecker chemistry, which is typically used to produce amino acids, results in a racemic mixture of nitrites which are converted to amides via a non-enantioselective nitrile hydratase and then converted to an amino acid via an enantioselective amidase. The unconverted amides must then be converted back to a racemic mixture of nitrites in a laborious procedure.

To overcome this problem, various enantiospecific nitrile hydratases and nitrilases have been isolated, e.g., from microorganisms. For example, WO 86/07386, published Dec. 18, 1986 by Godtfredsen et al., describes isolation of a naturally occurring S-selective nitrilase and nitrile hydratase. However, the enantiomeric excess is only about 40%. WO 92/05275, published Apr. 2, 1992 by Anton et al., describes the production of enantiomeric alkanoic acids using enantioselective nitrile hydratases. However, the hydratases isolated convert less than 10 mM of substrate over a 48 hour period and do not yield a high enantiomeric excess. The isolated enzymes are, therefore, insufficient for commercial processes.

These processes for making amino acids and other carboxylic acids and amide compounds are laborious and products obtained often may not have satisfactory enantiomeric purity. In addition, the processes are not typically efficient enough for most industrial processes.

New or improved methods of making amino acids, carboxylic acids, and amide compounds are, accordingly, desirable, particularly those that are amenable to industrial manufacturing techniques. The present invention fulfills these and other needs that will become apparent upon complete review of the following.

### SUMMARY OF THE INVENTION

The present invention provides methods of making amino acids, other carboxylic acids, and amides, using artificially evolved enantioselective enzymes such as nitrilases and nitrile hydratases. These nitrilases and nitrile hydratases typically have new or improved activity relative to currently available enzymes. For example, an R-selective nitrilase is optionally used to convert a racemic mixture of an amino nitrile to a mixture comprising an R-amino acid and unconverted S-amino-nitrile. Nitrile hydratases are used in the same manner to produce an amide which is then optionally converted to an amino acid using an amidase. In addition, methods of producing enantioselective enzymes and compositions comprising enantioselective enzymes are provided.

In one aspect, a method of converting a nitrile to an amide is provided. The method comprises contacting a nitrile, e.g., an amino nitrile, with an artificially evolved enantioselective, e.g., R-selective or S-selective, nitrile hydratase, thereby forming the amide. The nitrile typically comprises a racemic mixture, which when incubated with an enantioselective enzyme, yields a mixture comprising an optically active amide and unconverted nitrile.

The unconverted nitrile, e.g., S-nitrile when the enzyme used is R-selective, is optionally racemized, e.g., chemically, to produce another racemic nitrile mixture. The racemic mixture is then incubated with the enantioselective nitrile hydratase to produce more of the desired optically active amide.

In one embodiment, the nitrile hydratases of the invention are used to make amino acids, e.g., optically active amino acids. Making amino acids using the methods of the invention typically comprises contacting an amino nitrile with an artificially evolved enantioselective nitrile hydratase, thereby producing an amide. When a racemic mixture of amino nitrites is used as the substrate, the enantioselective hydratases of the invention convert only one enantiomer, e.g., the R-amino nitrites or the S-amino nitrites, to amides and leave the other enantiomer unconverted. The optically active amide is then typically contacted with an amidase, e.g., a non-selective amidase, to form an amino acid. The unconverted nitrile enantiomer is optionally racemized and then subjected to the enantioselective conversion reaction again.

In another aspect, methods are provided to convert a nitrile, e.g., an amino nitrile, directly to a carboxylic acid, e.g., an amino acid. The method comprises contacting a nitrile with an artificially evolved enantioselective nitrilase, thereby forming the carboxylic acid, e.g., an R-amino acid or an S-amino acid. When a racemic mixture is used as a substrate in an enantioselective reaction, the product is an optically active carboxylic acid and unconverted nitrile. As described above, the unconverted nitrile is optionally racemized and then re-subjected to the enantioselective reaction.

In another aspect, the present invention provides methods of converting a first enantiomer of a target molecule to a second enantiomer of the target molecule. The methods typically comprise converting the first enantiomer of the target molecule to a first enantiomer of an activated target molecule or a racemic mixture of the activated target molecule. The activated target molecule is then contacted with a racemase and an enantioselective enzyme, e.g., a fusion enzyme comprising a racemase and an enantioselective esterase or amidase. In some embodiments, the enzymes are artificially evolved enzymes, e.g., used to convert an L-amino acid to a D-amino acid. Typical target molecule comprises a carboxylic acid, for which the activated target molecule is optionally the corresponding ester. Other target molecules include, but are not limited to, amino acids, esters, amines, alcohols, and the like.

The racemase continuously converts the first enantiomer of the activated target molecule to a racemic mixture of the activated target molecule and the enantioselective enzyme converts the second enantiomer of the activated target molecule to the second enantiomer of the target molecule. For example, an ester is continuously racemized to a 1:1 mixture of its enantiomers, while the desired enantiomer is continuously converted to the corresponding carboxylic acid by an esterase.

The artificially evolved enzymes of the invention, e.g., an R-selective nitrilase, an R-selective nitrile hydratase, an S-selective nitrilase, an S-selective nitrile hydratase, or the like, are typically produced by recombining two or more nucleic acids encoding a parental enzyme, e.g., a non-enantioselective nitrilase or nitrile hydratase, and/or by mutating one or more enzyme encoding a parental enzyme, e.g., in one or more cycles of recombination or mutation. Alternatively, the enantioselective nitrile hydratase is produced by error prone PCR or assembly PCR. One or more round of recombination/mutation is typically followed by one or more round of selection for the activity of interest. This process of recombination/mutation and selection can be repeated one or more times to improve one or more activity of interest.

Recombining two or more nucleic acids is typically performed using recursive recombination, whole genome recombination, synthetic recombination, in silico recombination, or the like. For example, two or more nucleic acids corresponding to the following Genbank accession numbers: M60264, X64359, E03179, X64360, D14454, M74531, AF257489, E08304, D90216, and E13931, or fragments thereof, are optionally recombined to provide an enantioselective nitrile hydratase. Two or more nucleic acids corresponding to the following Genbank accession numbers: D12583, D67026, L32589, D13419, E01313, and AB028892, or fragments thereof, are optionally recombined to provide an enantioselective nitrilase.

Mutating one or more nitrile hydratase or nitrilase nucleic acid is typically performed using site directed mutagenesis, cassette mutagenesis, random mutagenesis, recursive ensemble mutagenesis, in vivo mutagenesis, or other available methods. For example one or more nucleic acid corresponding to the following Genbank accession numbers: M60264, X64359, E03179, X64360, D14454, M74531, AF257489, E08304, D90216, and E13931, is optionally mutated to provide an enantioselective nitrile hydratase. Mutating one or more nucleic acid corresponding to the following Genbank accession numbers: D12583, D67026, L32589, D13419, E01313, and AB028892 is used to provide an enantioselective nitrilase of the invention.

For example, a nucleic acid encoding an enantioselective nitrilase or an enantioselective nitrile hydratase is optionally produced by providing a population of DNA fragments encoding at least one parental nitrilase or nitrile hydratase. Parental nitrilases and nitrile hydratases are optionally chosen from those listed above. The DNA fragments are then typically recombined to produce a library of recombinant DNA segments. These steps are optionally repeated. The library of recombinant DNA segments is then typically screened to identify recombinant DNA segments that encode an artificially evolved enantioselective nitrilase or enantioselective nitrile hydratase, e.g., R-selective or S-selective enzymes. The entire method is optionally repeated, e.g., using the enantioselective enzymes obtained as parental enzymes.

Screening or selecting the evolved enzymes typically comprises contacting a racemic mixture of a nitrile with the artificially evolved enantioselective nitrilase or nitrile hydratase to produce a product, e.g., a carboxylic acid or an amide. The product is then typically separated, e.g., from unconverted substrate and/or from other enantiomers. Separation is typically performed using HPLC or electrophoresis, e.g., capillary electrophoresis. In other embodiments, the product is analyzed using NMR which yields separate peaks for substrate and products, e.g., one peak for each enantiomer. Mass spectrometry is also used for screening, e.g., to screen whole cells for nitrilase or nitrile hydratase activity. Those cells identified as having, e.g., nitrilase activity, are then screened for enantioselectivity, e.g., using capillary electrophoresis or NMR.

For example, an enzyme that is not enantioselective, i.e., non-selective, yields a racemic mixture of product. However, an enantioselective enzyme yields an excess of one enantiomer. In addition, an enantioselective enzyme typically leaves some unconverted substrate, e.g., the enantiomer for which it does not select. An enantiospecific enzyme essentially converts only one enantiomer and leaves the other enantiomer of the substrate unconverted. The percentage of product, e.g., a carboxylic acid or amide of interest, is typically determined, e.g., the percentage of product comprising an R-enantiomer or the percentage of the product comprising an S-enantiomer. One or more artificially evolved enantioselective nitrilase or nitrile hydratase is optionally identified using the percentages determined, e.g., an enzyme that produced an excess of about 90% or more of the enantiomer of interest, e.g., an R-carboxylic acid, an S-carboxylic acid, an R-amide, or an S-amide. In other embodiments, the enantioselective nitrilases and nitrile hydratases of the invention produce about 95% or more, about 99% or more, or about 99.5% or more of the enantiomer of interest, e.g., an R-carboxylic acid, an S-carboxylic acid, an R-amide, or an S-amide.

Recombinant nitrilases or nitrile hydratases produced by the above methods are also embodiments of the present invention as well as compositions comprising them. In additional embodiments, reaction mixtures comprising the enzymes of the invention are provided. For example, in one embodiment, a reaction mixture comprising an amino nitrile, e.g., a racemic mixture, and an R-selective nitrile hydratase, an R-selective nitrilase, an S-selective nitrile hydratase, or an S-selective nitrilase is provided, e.g., artificially evolved enantioselective nitrilases and nitrile hydratases produced as described above. The reaction mixtures of the invention also optionally comprise an amidase and/or amide. In some embodiments, the reaction mixtures further comprise an R-amino acid, an S-amino acid, or an amide, e.g., an R-amide or an S-amide.

### BRIEF DESCRIPTION OF THE FIGURES

FIG. 1: Schematic for an enantioselective enzymatic reaction converting a racemic mixture of a nitrile to an optically active amide. The amide is then converted to a carboxylic acid, e.g., via a non-selective reaction, and the unconverted nitrile is racemized and subjected to the enantioselective reaction again.

FIG. 2: Schematic illustrating the reaction of FIG. 1 when used to produce D-phenylglycine.

FIG. 3: Schematic illustrating 100% conversion of a racemic mixture to chiral products.

FIG. 4: Schematic illustration of a 100% conversion of L-amino acids to D-amino acids.

### DETAILED DISCUSSION OF THE INVENTION

The present invention provides enantioselective methods of converting a nitrile to an amide or to a carboxylic acid. In one aspect, the invention provides an enantioselective method for converting a racemic mixture of amino-nitriles to R-amino acids or to S-amino acids. These methods provide improved routes to produce each of the amino acid enantiomers.

An “enantioselective” process or enzyme is one that yields an excess of one enantiomer (i.e., more than 50%, and typically less than about 100%). An “enantiospecific” process or enzyme yields one enantiomer almost exclusively. Such enantioselective enzymes are useful when catalyzing reactions or conversions to compounds comprising a chiral center. A “chiral center,” as used herein, refers to a carbon atom with four different point ligands or groups attached thereto. In the present invention, enantioselective reactions typically result in an excess of about 60% or more, preferably about 90% or more of one enantiomer, more preferably about 95% or more, about 99% or more or about 99.5% or more.

For example, an R-selective or S-selective process or enzyme is one that results in about 60% to about 90% or more of an R-enantiomer or S-enantiomer respectively. For example, in the present invention a racemic mixture of nitrile compounds is typically used as a substrate for an enantioselective enzyme. An enantioselective enzyme converts the nitrites to an amide or a carboxylic acid, depending on the type of enzyme used, comprising about 60% or more or about 90% or more of one enantiomer. For example, an R-selective nitrilase converts nitrites, e.g., R-nitriles in a racemic mixture, to R-carboxylic acids. An S-selective nitrie hydratase converts nitrites to S-amides. R-specific or S-specific are used to refer to enzymes or processes that yield an R or S product exclusively. The present invention includes R-selective, R-specific, S-selective, and S-specific enzymes for the conversion of nitrites, e.g., amino nitrites, to amides and carboxylic acids, e.g., amino acids.

In addition to describing processes and enantioselective enzymes, the R and S designations are used to refer to particular enantiomers of chiral compounds, e.g., the amides, carboxylic acids, and nitrites of the invention. A chiral molecule typically comprises a carbon atom with four different groups attached to it. A chiral molecule does not have a plane of symmetry and can be represented by two nonsuperimposable mirror image structures, known as enantiomers. The two enantiomers rotate a plane of polarized light in opposite directions but with equal magnitudes. An enantiomer that rotates the light in a clockwise direction is designated as a D-enantiomer. An enantiomer that rotates light in a counterclockwise direction is designated as an L-enantiomer. A chiral compound composed primarily of one enantiomer rotates a plane of polarized light and is, therefore, optically active. A chiral compound which is composed of substantially equal mixtures of the enantiomers does not rotate a plane of polarized light and is said to be optically inactive. Such mixtures are called racemic mixtures.

Another designation system that is commonly used to name enantiomers is an absolute configuration system in which each group attached to the chiral center, e.g., the carbon, is assigned a number or priority. Methods of assigning numbers and/or priority are well known to those of skill in the art. For a review of chirality, see, e.g., Organic Chemistry, Fourth Edition, by Pine et al., McGraw Hill, New York (1980); and Organic Chemistry, Second Edition, by Fessendon and Fessendon, Willard Grant Press, Boston (1982). Starting at number one and proceeding to 4 in a chiral molecule represents either a clockwise or counterclockwise rotation of the molecule. A clockwise rotation leads to the designation R and a counterclockwise rotation leads to the designation S. In the present invention, the nitrites, amides, and carboxylic acids are designated R or S. Nitrilases and nitrile hydratases that act on or catalyze hydrolysis or hydration of predominantly one enantiomer, e.g., an excess of about 90% or more R or S enantiomer, are provided herein and used, e.g., to produce optically active amino acids.

A nitrile as used herein refers to a compound comprising a cyano group (CN), e.g., a compound having Formula I or Formula II:

R—CN I

II

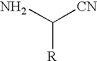

wherein R comprises any organic compound or substituent. Typically R comprises an alkyl, e.g., lower alkyl comprising about 1 to about 10 carbon atoms, or a phenyl group, e.g., hydroxyphenyl. In addition, an alkyl group of the invention is optionally a substituted alkyl group, e.g., substituted with a hydroxy group, a halogen, sulfur, an amino group, a phenyl group, a carboxy group, an alkoxy group, a phosphate group, or the like. Formula II comprises an amino nitrite of the invention. The amino nitrites of the invention are typically used to produce amino acids, e.g., D-amino acids or L-amino acids.

An “amide” is used herein to refer to a compound comprising a trivalent nitrogen attached to a carbonyl group. Typical amides of the invention include compounds having Formula III or Formula IV:

R—CONH₂ III

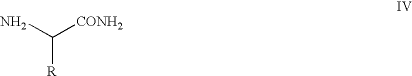

wherein R includes any organic compound or substituent as described above.

A “carboxylic acid” of the invention is a compound having a carboxyl group, e.g., as shown in Formulas V and VI:

R—COOH V

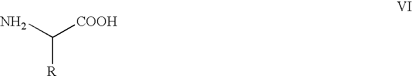

wherein R is defined as above, e.g., any organic compound or constituent, substituted or unsubstituted. Formula VI represents an amino acid of the invention.

“Amino acid” as used herein includes any naturally or non-naturally occurring amino acids as well as modified amino acids. An amino acid typically comprises an amino group, a carboxyl group, a hydrogen atom and an R group all bonded to a carbon atom which forms a chiral center and is called the α-carbon. Naturally occurring amino acids include, e.g., glycine, valine, alanine, leucine, isoleucine, phenylalanine, tyrosine, tryptophan, cysteine, methionine, serine, threonine, lysine, arginine, histidine, aspartate, glutamate, asparagine, and glutamine. Modified amino acids include, but are not limited to, hydroxyproline, γ-carboxyglutamate, O-phosphoserine, O-phosphotyrosine, and the like. Modified amino acids are well known to those of skill in the art and are included within the scope of the present invention. Typical amino acids of the invention are represented by Formula VI.

The chiral center in an amino acid confers optical activity on amino acids. The two mirror image forms of the amino acids are typically referred to as an L-isomer and a D-isomer. Only L-isomers are constituents of proteins. In fact, most amino acids found in nature are of the L-configuration. Therefore, for food, food additives, and drugs, it is often desirable to produce pure L-amino acids because D-enantiomers are not typically metabolizable by living cells. However, the fact that they are not metabolized and can interfere with normal cell metabolism and cell function makes D-amino acids useful in some instances. For example, incorporation of such amino acids in pharmacologically active compounds can lead to enhanced activity due to unnatural chirality. In these instances, it also important that the amino acid used is an optically active D-enantiomer. Therefore, the present invention is optionally used to produce L-amino acids or D-amino acids, e.g., in enantiomerically pure forms. In general, the S-designation described above typically corresponds to L-amino acids and the R-designation described above typically corresponds to D-amino acids.

The present invention provides methods of producing chiral amino acids by providing enantioselective nitrilases and nitrile hydratases, e.g., enzymes that distinguish between two enantiomers. “Nitrile hydratase” is used herein to refer to a polypeptide, protein, or other enzyme that catalyzes the hydration of a nitrile to the corresponding amide. For example, a nitrile hydratase catalyzes the hydration of an amino nitrile, e.g., of Formula II, to an amide, e.g., of Formula IV. An enantioselective nitrile hydratase exhibits a strong selectivity towards one enantiomer. Therefore, the amides produced by the nitrile hydratases of the invention comprise an excess of one of the two enantiomers. Typically, the excess is about 50% or greater. Preferably, the amides of the invention are produced in an excess of about 90% or greater. More preferably, the excess is about 95% or greater, about 99% or greater or about 99.5% or greater. When producing amino acids, the methods of the present invention use nitrile hydratases to produce an amide in enantiomeric excess which is then converted to the amino acid, e.g., via a non-selective amidase. Any unconverted nitrile can be racemized and subjected to reaction with the enantioselective enzyme again.

“Enantiomeric excess” is defined herein as the percentage of one enantiomer produced minus the percentage of the opposite enantiomer produced in a process, e.g., in the production of an amino acid using an enantioselective nitrilase. Therefore, if 90% of the S-form of phenylglycine is produced and 10% of the R-form is produced, the enantiomeric excess is 90−10=80%. Amounts of both enantiomers are typically detected and the equation used to calculate the enantiomeric excess is (A−B)/(A+B)×100%.

Another method for producing amino acids comprises contacting a nitrile, e.g., an amino nitrile, with a nitrilase. “Nitrilase” is used herein to refer to a polypeptide, protein, or other enzyme that catalyzes the conversion of a nitrile to a carboxylic acid. The nitrilases of the invention are therefore used to produce an amino acid directly from a nitrile. When an enantioselective nitrilase is used, an optically active amino acid is optionally prepared. An enantioselective nitrilase exhibits a strong selectivity towards one nitrile enantiomer. Therefore, the carboxylic acids produced by the nitrilases of the invention comprise an excess of one of the two enantiomers. Typically, the excess is about 50% or greater. Preferably, the carboxylic acids of the invention are produced in an excess of about 90% or greater. More preferably, the excess is about 95% or greater, about 99% or greater or about 99.5% or greater. When producing amino acids, the methods of the present invention typically use nitrilases to produce an amino acid in enantiomeric excess. Any unconverted nitrile can be racemized and subjected to reaction with the enantioselective nitrilase again.

“Artificially evolved” enzymes, e.g., nitrilases or nitrile hydratases, are protein based catalysts or enzymes generated using, e.g., DNA shuffling or recursive recombination, mutagenesis, and the like. Chimeric enzymes that include identifiable component sequences derived from two or more parents can also be used. When a particular product includes enantiomeric isomers, artificially evolved nitrilases or nitrile hydratases typically yield the amide or carboxylic acid in an enantioselective manner. They can also be evolved to yield those products enantiospecifically. For example a nitrilase is optionally artificially evolved to enantiospecifically or enantioselectively cataylze the conversion of an amino nitrile to an R-amino acid.

The artificially evolved enzymes of the invention, e.g., an R-selective nitrilase, an R-selective nitrile hydratase, an S-selective nitrilase, an S-selective nitrile hydratase, or the like, are typically produced by recombining, e.g., using recursive recombination, whole genome recombination, synthetic recombination, in silico recombination, or the like, two or more nucleic acids encoding a parental enzyme, e.g., a non-enantioselective nitrilase or nitrile hydratase, or by mutating one or more nitrile hydratase or nitrilase nucleic acid, e.g., using site directed mutagenesis, cassette mutagenesis, random mutagenesis, recursive ensemble mutagenesis, or in vivo mutagenesis. Alternatively, the enantioselective nitrile hydratase is produced by error prone PCR or assembly PCR. A nucleic acid encoding a parental enzyme refers to a nucleic acid or gene that, through the mechanisms of transcription and translation, produces an amino acid sequence corresponding to a parental enzyme, e.g., a naturally occurring nitrilase or nitrile hydratase in the present invention.

Typically, chiral amino acids are produced using Strecker chemistry to produce a racemic α-amino nitrile. The nitrile is then converted to the amide by a non-selective nitrile hydratase, and then selectively converted to the chiral amino acid via an enantioselective amidase. The unreacted amide may then be converted to the nitrile, racemized, and the process repeated. One disadvantages of this method relates to the complexity of the chemical process by which the amide is dehydrated to the amino nitrile. The process requires 3-steps and is inefficient and expensive. As a result, other chemistries, such as hydantoin-based intermediates, are favored for large-scale economic production of some amino acids such as D-phenylglycine. However, use of hydantoinases is limited to the synthesis of D-enantiomers. In addition, they are oxygen sensitive, have poor water solubility, and cannot hydrolyze di-substituted cyclic amides.

Alternatively, an enantioselective enzyme as described above, e.g., one artificially evolved as described in more detail below, is used to simplify the production of amino acids. The resulting process begins with an enzymatic conversion of an amino nitrile to a single enantiomer of an amide. A non-specific amidase is then optionally used to convert the amide to the amino acid. This eliminates the complex step of converting unused amide to nitrile. Instead, the unreacted nitrile is merely racemized in a simple, one step procedure and the enantioselective process is repeated. In other embodiments, a direct conversion of the nitrile to the amino acid can be made using an enantioselective nitrilase. Industrial production of amino acids is made easier, more efficient, and less expensive by allowing enantioselective production, e.g., via artificially evolved nitrilase and/or nitrile hydratases. Although naturally occurring S-selective nitrilases and nitrile hydratase have been identified (See, e.g., WO 86/07386 and WO 92/05275), the R-amino acids have not previously been easy or efficient to produce, e.g., on an industrial level. The present methods provide easy and efficient methods for the production of enantiomerically pure amino acids, e.g., R-amino acids.

I. Enzymatic Conversion of an Amino Nitrile to an Amide

The present invention provides methods of converting a nitrile to its corresponding amide, e.g., an amino nitrile to an amide. For example, a nitrile of Formula I is optionally converted to an amide of Formula III, e.g., using an artificially evolved nitrile hydratase. An amino nitrile of Formula II, is optionally converted to an optically active amide of Formula IV using a nitrile hydratase of the invention.

To convert a nitrile to an amide using a nitrile hydratase, the nitrile is contacted, e.g., in aqueous solution, with the nitrile hydratase, e.g., using an isolated or recombinant form of the enzyme or one or more cells that possess nitrile hydratase activity. For example, a racemic mixture of amino nitrites is incubated with an enantioselective nitrile hydratase, e.g., by stirring a mixture comprising the nitrile hydratase and the nitrile in an aqueous solution, e.g., for about 1 to about 25 hours or more typically about 4 to about 25 hours or about 4 to about 12 hours. Reaction temperatures, pH, salt concentrations, and incubation times are optionally varied. Typical temperatures range from about 4° C. to about 70° C., or about 4° C. to about 37° C. Typical pHs range from about 5 to about 8. Organic solvents are optionally added, e.g., to increase the solubility of the reactants.

Nitrile hydratases used in the present invention are typically artificially evolved enzymes, e.g., in a purified form, in a crude enzyme solution, in microbial cells exhibiting nitrile hydratase activity. For example, hydration of a nitrile to an amide by a nitrile hydratase is conveniently carried out using cells, e.g., microbial or bacterial cells, that possess sufficient activity of one or more nitrile hydratase that acts on nitrites enantioselectively as substrates, e.g., cells that have been transformed with a nucleic acid encoding an artificially evolved nitrile hydratase. Artificially evolved nitrile hydratases are described in more detail below.

Various microorganisms are optionally used to carry out the conversion, including, but not limited to, bacteria, cyanobacteria, fungi, yeasts, and the like. A preferred embodiment uses bacterial strains. Various bacterial strains are optionally used for the purpose, including E. coli and other species selected from the following non-limiting examples of genera of known microorganisms: Pseudomonas, Rhodococcus, Burkholderia, Sphingomonas, Comamonas, Alcaligenes, Acinetobacter, Bacillus, and the like. E. coli is typically used because this organism is generally recognized as safe in biotechnological applications. Other, e.g., non-pathogenic, species are also optionally used. The strains are optionally prototrophic or auxotrophic in respect to different growth requirements and nutrients, and the bacterial cells can be grown in a variety of media of defined or undefined compositions well known in the art.

The nitrile hydratase, when it contacts the nitrite mixture, catalyzes the hydration of the nitrite to an amide. An enantioselective nitrile hydratase distinguishes between the R-nitriles and S-nitriles and converts one enantiomer preferably over the other. An R-selective nitrite hydratase typically yields an R-amide, e.g., in enantiomeric excess of about 60% to about 90% or more over the S-amide, mixed with unconverted S-nitrile.

The amide enantiomer of interest and the unconverted nitrite produced as described above are typically separated, e.g., by neutralization and solvent extraction. The R-amide is optionally used in further steps, e.g., to produce a carboxylic acid. The unconverted S-nitrile, e.g., that portion of the racemic mixture which is not converted by an enantioselective enzyme, is optionally racemized into a mixture of the R-nitrile and the S-nitrile and then subjected to the hydration step to produce more R-amide. In this manner, an enantiomerically pure compound, e.g., about 90% or more of a single enantiomer, is optionally prepared.

Racemization of the unconverted nitrile enantiomer, e.g., an S-nitrile, is optionally performed to recycle any unconverted nitrile, e.g., to further the conversion of an initial racemic nitrile mixture into an enantiomerically pure amide. Racemization is optionally carried out enzymatically, e.g., using a naturally occurring or artificially evolved racemase, or chemically, e.g., using a basic ion exchange resin in an organic solvent. The resulting racemic mixture of nitrile is then optionally hydrolyzed using an enantioselective or enantiospecific nitrile hydratase as described above.

Reaction mixtures as used above are also embodiments of the present invention. For example, the invention provides a reaction mixture comprising an artificially evolved enantioselective nitrile hydratase and a nitrile, e.g., an amino nitrile. The nitrile is typically a racemic mixture. In addition, the reaction mixtures of the invention optionally comprise an amide, e.g., as produced above. An example reaction mixture of the present invention optionally comprises an R-selective nitrile hydratase, an S-nitrile, and an R-amide.

The amides of the invention are optionally used to produce amino acids, e.g., R-amino acids, as described in more detail below.

II. Enzymatic Conversion of an Amide to an Amino Carboxylic Acid

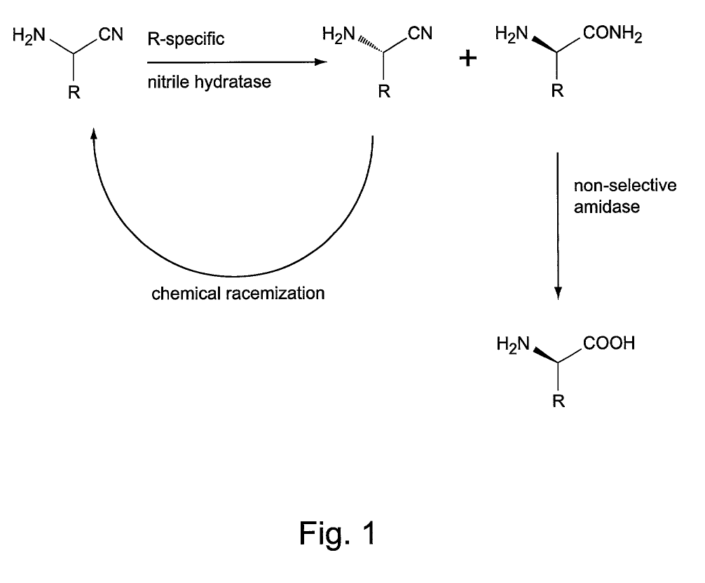

Amides, e.g., produced as described above are optionally converted to amino acids or other carboxylic acids, e.g., enantiomerically pure carboxylic acids. The amide enantiomer of interest, e.g., separated from undesirable enantiomers or unconverted substrates by HPLC, is optionally converted to the corresponding acid using an amidase, e.g., a non-selective amidase that converts all amide configurations to acids substantially equally. See, e.g., FIG. 1. Alternatively, an amidase that is selective for the enantiomer of interest is optionally used. Alternatively, the amides produced as described above are chemically converted into carboxylic acids, e.g., using techniques well known to those of skill in the art such as techniques involving a mineral acid.

To convert an amide of the present invention to a carboxylic acid, the amide, e.g., an R-amide or an S-amide, is typically contacted with an amidase, e.g., in a purified form or crude cell extract. The amidase is optionally an isolated naturally occurring amidase or an artificially evolved amidase. The incubation is optionally carried out in the same manner as the enzymatic conversion described above. A non-selective amidase is sufficient to produce an enantiomerically pure carboxylic acid in the present invention because the amide produced from the enantioselective nitrile hydratase is typically an optically active enantiomer. Alternatively, an appropriate enantioselective amidase is also optionally used.

If the amidase reaction is carried in the same solution as that used for the creation of the amide, e.g., concurrently, the final product is typically an unconverted nitrile enantiomer and a carboxylic acid of the desired enantiomer, e.g., in excess of about 90% or more. The nitrile and the carboxylic acid are optionally separated, e.g., using HPLC, and the nitrile is recycled, e.g., racemized and subjected to enantioselective hydration.

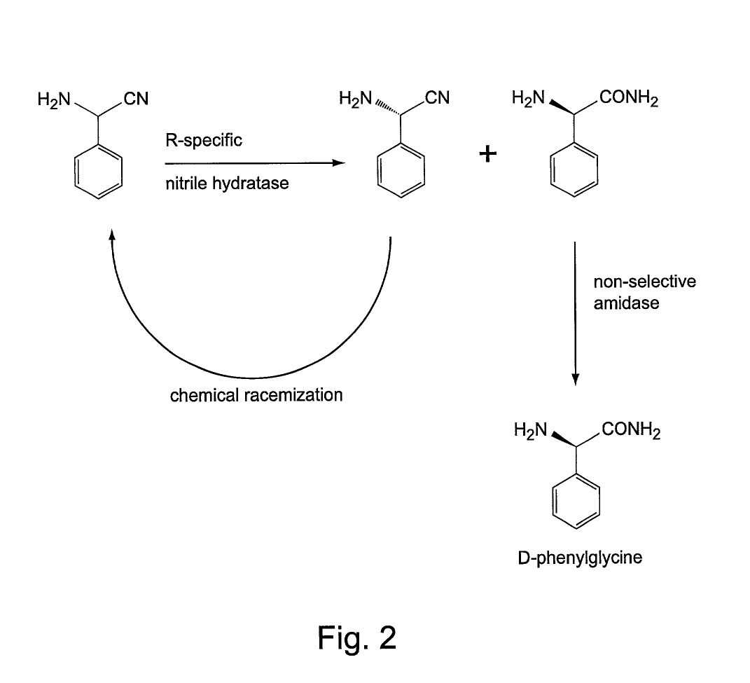

For example, D-phenylglycine and 4-hydroxy-phenylglycine are optionally produced, as shown in FIG. 2, using an artificially evolved enantioselective nitrile hydratase. R-phenylglycine and 4-hydroxy-phenylglycine are two intermediates used in the production of semi-synthetic penicillins, and are optionally produced using the methods of the invention. The racemic nitrile mixture is easily synthesized using methods known to those of skill in the art, e.g., from benzaldehyde, ammonia, and HCN, e.g., using Strecker chemistry. An R-selective nitrile hydratase generates predominantly the R-amide, which is then converted to the R-enantiomer of the product by acid or enzyme hydrolysis. See, e.g., FIG. 2. Following separation of the unconverted amino nitrile from the product, the unconverted amino nitrile is then optionally racemized and recycled. Alternatively, the racemization is performed under conditions in which the nitrile hydratase is active, e.g., evolved to be active, thus allowing a continuous, essentially complete conversion of the nitrile to the desired amide enantiomer and/or carboxylic acid enantiomer.

Alternatively, a non-specific nitrile hydratase is used to convert the nitrile of interest to an amide, e.g., a racemic mixture of the amide. An enantioselective amidase then converts one of the amide enantiomers to a carboxylic acid of interest. A racemase is optionally used in the same solution to continuously convert any unconverted amide to a racemic mixture to eventually produce an optically active carboxylic acid. This is explained in more detail below.

III. Enzymatic Conversion of a Nitrile to Carboxylic Acid

In other embodiments, a nitrile is converted directly to a carboxylic acid using a single enzymatic conversion. A nitrilase converts a nitrile, e.g., an amino nitrile, to a carboxylic acid, e.g., an amino carboxylic acid. In the present invention nitrilases are optionally evolved to be enantioselective, e.g., R-selective or R-specific, thereby allowing the production of enantiomerically pure R-amino acids. The enantioselective nitrilases of the invention typically produce an enantiomeric excess about 60% to about 90% or more of the desired enantiomer. Preferably the enantiomeric excess is about 95% or more, and more preferably about 99% or more.

To convert a nitrile to a carboxylic acid using a nitrilase, the nitrile is contacted, e.g., in aqueous solution, with the nitrilase, e.g., using an isolated or recombinant form of the enzyme or one or more cells that possess nitrilase activity. For example, a racemic mixture of amino nitrites is incubated with an enantioselective nitrilase, e.g., by stirring a mixture comprising the nitrilase and the nitrile in an aqueous solution, e.g., for about 10 minutes to about 25 hours, typically about 1 to about 25 hours, or about 5 to about 20 hours, or more typically, about 5 to about 15 hours. Reaction temperatures, pH, incubation times, and other reaction conditions are optionally varied. A typical pH for such a reaction is below pH 8, preferably between about 5 and about 8, and more preferably between about 5 and 7. Typical temperatures range from about 4° C. to about 70° C., more typically about 4° C. to about 45° C., and most typically about 4° C. to about 37° C. Organic solvents are optionally added, e.g., to increase the solubility of the reactants. Typical substrate concentrations are at least about 10 mM, preferably, at least about 50 mM, and more preferably at least about 100 mM or more. For example, 20 mM phenylglycine nitrile is optionally converted to phenylglycine in about an hour using the nitrilases of the invention. In other embodiments, the concentration of the nitrile substrate is about 1 M or more, preferably about 1.3 M or greater.

Nitrilases used in the present invention are typically artificially evolved enzymes, e.g., in a purified form, in a crude enzyme solution, or in microbial cells exhibiting nitrile hydratase activity. For example, conversion of a nitrile to a carboxylic acid by a nitrilase is conveniently carried out using cells, e.g., microbial or bacterial cells, that possess sufficient activity of one or more nitrilase that acts on nitrites enantioselectively as substrates, e.g., cells that have been transformed with a nucleic acid encoding an artificially evolved nitrilase. Alternately, the conversions can be performed in vitro, i.e., with compositions comprising the relevant enzymes and products. Artificially evolved nitrilases are described in more detail below.

Various microorganisms are optionally used to carry out the conversion, including, but not limited to, bacteria, cyanobacteria, fungi, yeasts, and the like. A preferred embodiment uses bacterial strains as described above. Various bacterial strains are optionally used for the purpose, including E. coli and other species selected from the following non-limiting examples of genera of known microorganisms: Pseudomonas, Rhodococcus, Burkholderia, Sphingomonas, Comamonas, Alcaligenes, Acinetobacter, Bacillus, and the like.

The nitrilase, when it contacts the nitrite mixture, catalyzes the hydrolysis of a nitrite to a carboxylic acid. An enantioselective nitrilase distinguishes between the R-nitriles and S-nitriles and converts one enantiomer preferably over the other. An R-selective nitrilase typically yields an R-carboxylic acid, e.g., in enantiomeric excess of about 90% or more over the S-carboxylic acid, typically mixed with unconverted S-nitrile.

The carboxylic acid enantiomer of interest and the unconverted nitrite produced as described above are typically separated, e.g., by neutralization and solvent extraction. The unconverted nitrite enantiomer, e.g., that portion of the racemic mixture which is not converted by an enantioselective enzyme, is optionally racemized into a mixture of the R-nitrile and the S-nitrile and then subjected to the hydrolysis step to produce more of the carboxylic enantiomer of interest, e.g., an R-amino acid or an S-amino acid.

Racemization of the unconverted nitrile enantiomer, e.g., the S-nitrile, is optionally performed to recycle the unconverted nitrite, e.g., to further the conversion of an initial racemic nitrite mixture into an enantiomerically pure carboxylic acid. Racemization is optionally carried out enzymatically, e.g., using a naturally occurring or artificially evolved racemase, or chemically, e.g., using a basic ion exchange resin in an organic solvent. The resulting racemic mixture of nitrite is then optionally hydrolyzed using an enantioselective or enantiospecific nitrite hydratase as described above. The racemization is optionally carried out as a separate step, e.g., after separating the unconverted nitrite from the reaction mixture, or it is carried out in the same reaction vessel, e.g., continuously racemizing any unconverted nitrite to maintain a 1:1 ratio of nitrite enantiomers.

In another embodiment, a one pot synthesis of enantiomerically pure amino acids is obtained using a reversible reaction to produce an amino nitrite. For example, a ketone or aldehyde is reacted with ammonia and potassium cyanide to produce the corresponding amino nitrile. The amino nitrile is then selectively converted to an amino acid, e.g., D or L, using an enantioselective nitrilase. Because the production of the nitrile from the aldehyde or ketone is reversible, production of a chiral amino acid is achieved with about a 60% to about a 90% or greater yield, preferably about a 95% or greater yield, or more preferably a 99% or greater yield. In the same manner, enantiomerically pure hydroxyl acids are also prepared, e.g., from aldehydes or ketones, in a single pot synthesis. Reaction of the aldehyde or ketone with potassium cyanide yields the hydroxyl nitrile which is then converted, using a nitrilase, to the corresponding acid. For more details, see the examples provided below. See, also, Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York).

Reaction mixtures as used above are also embodiments of the present invention. For example, the invention provides a reaction mixture comprising an artificially evolved enantioselective nitrilase and a nitrile, e.g., an amino nitrile. The nitrile is typically a racemic mixture. In addition, the reaction mixtures of the invention optionally comprise a carboxylic acid, e.g., an amino acid. For example a example reaction mixture of the present invention comprises an R-selective nitrilase, an S-amino nitrile, and an R-amino acid.

Methods for preparing artificially evolved enantioselective enzymes, e.g., nitrilases and nitrile hydratase, for use in production, e.g., industrial production, of enantiomerically pure or substantially pure amino acids are described in more detail below.

IV. Esterases and Hydrolases Used to Produce Enantiomerically Pure Amino Acids

In addition to nitrile hydratases and nitrilases, the present invention also provides for enantioselective racemases, enantioselective hydrolases, and enantioselective hydantoinases, e.g., for use in the production of enantiomerically pure compounds, e.g., R-compounds or S-compounds. For example, combinations of enantioselective racemases and hydrolases are optionally used for the production of D-amino acids, L-amino acids, and their chiral esters and amides. Libraries of these enzymes are optionally produced as described above and in more detail below and screened for function on any target compound, e.g., target amides, esters, and hydantoins, to determine enantiomeric excess. For example, evolved enzymes are typically screened for selective hydrolysis of one enantiomer of an activated form of a target chiral compound.

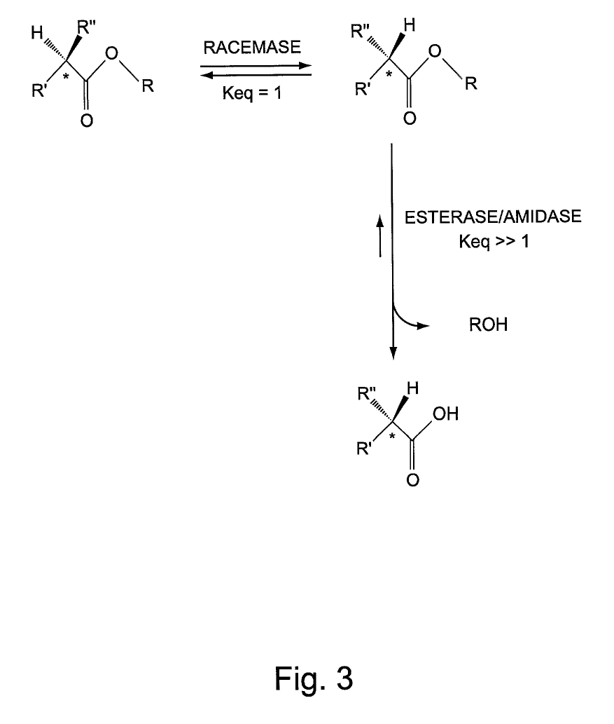

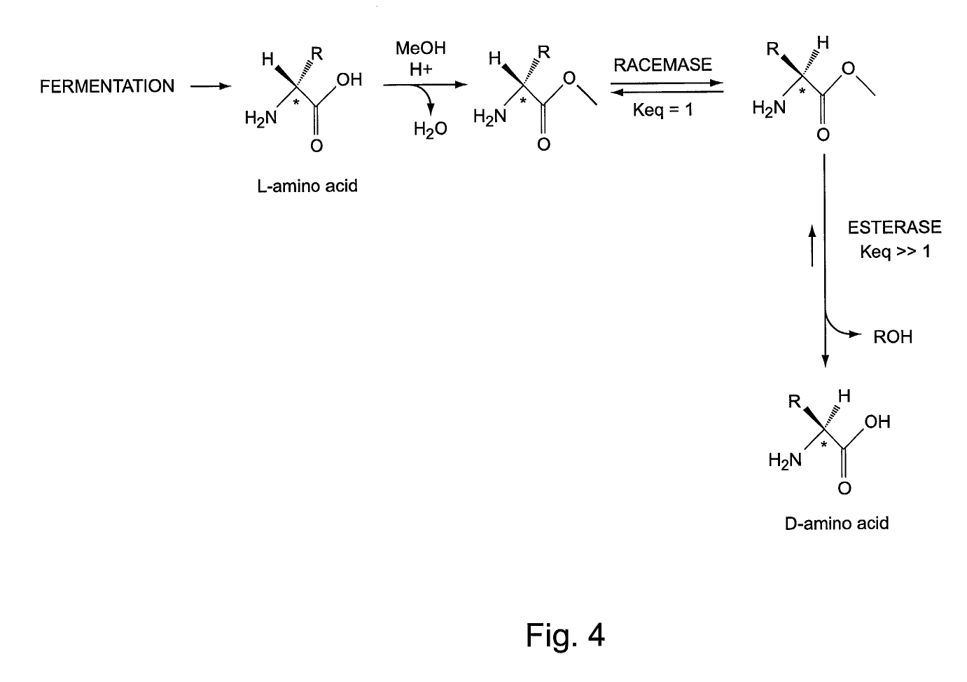

The methods presented herein are typically used to convert a first enantiomer of a target molecule to a second enantiomer of the target molecule, e.g., a carboxylic acid, an ester, an amine, an alcohol, or the like. For example, the methods involve converting the first enantiomer of the target molecule to an activated target molecule, e.g., a first enantiomer of the activated target molecule or a racemic mixture. In one embodiment, the target molecule comprises a carboxylic acid and the activated target molecule comprises the ester of the carboxylic acid. The activated target molecule is then incubated or contacted with a racemase and an enantioselective enzyme, e.g., as a fusion enzyme. The racemase continuously converts the first enantiomer of the activated target molecule to a racemic mixture comprising the first enantiomer of the activated target molecule and the second enantiomer of the activated target molecule; while the enantioselective enzyme converts the second enantiomer of the activated target molecule to the second enantiomer of the target molecule. For example, see, e.g., FIGS. 3 and 4.

Functional racemases and esterases are optionally combined, e.g., in a single pot, cell, or fusion enzyme, with the racemate of the target compound to be converted. For example, a hydrolytic enzyme enantioselectively hydrolyzes one form of the target, e.g., an ester. The racemase functions to continuously convert the non-hydrolyzed ester to the racemate. Over time, all of the ester is converted to a single enantiomer of the acid, alcohol, amine, carbamate, or other desired product.

These enzymes are also optionally used to complete conversion of one enantiomer of a target acid, alcohol, amine, or the like to its opposite enantiomer. For example, these enzymes are optionally used in the production of D-amino acids from L-amino acids, e.g., naturally isolated L-amino acids, as shown in FIGS. 3 and 4. FIG. 3 illustrates 100% conversion of a racemic mixture to a chiral product using a racemase and an esterase and/or amidase. FIG. 4 shows the conversion of L-amino acids, e.g., produced from fermentation, to D-amino acids using racemase and esterase.

For example, the production of D-tryptophan is optionally performed using enantioselective enzymes. A library of amino acid racemases, e.g., alanine racemases, is generated, e.g., through shuffling a family of genes or through any other diversity generation method. In addition, a library of ester hydrolases is also generated from shuffling a family of genes or from any other diversity generation protocol as described in more detail below. The methyl ester of L-tryptophan is produced, e.g., by condensation with methanol. Each member of the racemase library is then screened for a loss of enantiomeric excess and the production of the racemate from the L-tryptophan methyl ester. The ester hydrolase library is then typically screened, e.g., independently, for the selective hydrolysis of D-tryptophan methyl ester. An efficient racemase and an efficient D-selective tryptophan methyl ester hydrolase are then combined and mixed with pure L-tryptophan methyl ester or a D, L-tryptophan methyl ester racemate. The ester hydrolase selectively hydrolyzes the D-methyl ester. As the concentration of the D-methyl ester decreases, the racemase maintains a 1:1 equilibrium of the D and L enantiomers. Over time, the L-enantiomer is completely converted to the D-enantiomer with the reaction equilibrium being pulled by the energetically favorable release of methanol upon D-ester hydrolysis.

The methods described above provide complete conversion of a first enantiomer to a second enantiomer. For example, the reactions are typically continued until substantially all of the first enantiomer of the target molecule is converted into the second enantiomer of the target molecule, e.g., about 90% or more or 95% or more of the first enantiomer of the target molecule is converted into the second enantiomer of the target molecule.

V. Methods of Making Enantioselective Nitrilases and Nitrile Hydratases

The production of enantiomerically pure compounds, e.g., amino acids, is of great interest in many industries. Therefore, the present invention provides methods for enantioselectively producing amino acids, e.g., using artificially evolved nitrilases and nitrile hydratases. Methods for converting one enantiomer to its opposite enantiomer are also provided, e.g., using artificially evolved racemases, esterases, amidases and the like.

Wild-type nitrilases and nitrile hydratases as well as mutants, chimeras, and variants, are optionally used in the methods described above to enzymatically prepare amino acids. However, the enzymes previously known are not efficient in preparing enantiomerically pure amino acids, e.g., R-amino acids.

For example, s-selective nitrile hydratases isolated from Pseudomonas putida are optionally used to enzymatically convert nitrites to amides. See, e.g., U.S. Pat. No. 5,811,286. However, improved nitrile hydratases are also desirable, e.g., to provide higher levels of enantioselectivity, to provide R-selective enzymes, and to provide enzymes that convert amino-nitriles.

In one embodiment, the present invention provides a method of producing a nucleic acid encoding an enantioselective nitrilase or an enantioselective nitrile hydratase. The method comprises providing a population of DNA fragments encoding at least one parental enzyme, such as a nitrilase, nitrile hydratase, racemase, esterase, or the like. DNA fragments typically result from cleavage of at least one or more of the parental nucleic acids, e.g., chemical or enzymatic cleavage. Alternatively, DNA fragments are provided from subsequences of the parental nucleic acids produced in any other manner, including e.g., partial elongation of complementary sequences. DNA fragments include, but are not limited to, DNA, PCR amplicon fragments, synthetic oligonucleotides, and the like.

Parental nitrilases of the invention include, but are not limited to, nucleic acids corresponding to the following Genbank accession numbers: D12583, D67026, L32589, D13419, E01313, and AB028892. Parental nitrile hydratases of the invention include, but are not limited to, nucleic acids corresponding to the following Genbank accession numbers: M60264, X64359, E03179, X64360, D14454, M74531, AF257489, E08304, D90216, and E13931.

The DNA fragments are recombined to produce a library of recombinant DNA segments. A library, as used herein, refers to a set of polynucleotides. The set is optionally pooled or is individually accessible. The set is optionally made up of DNA, RNA or combinations thereof. These steps are optionally repeated to produce one or more libraries of recombinant DNA segments.

The library of recombinant DNA segments is then typically screened to identify at least one recombinant DNA segment that encodes an artificially evolved enantioselective nitrilase or enantioselective nitrile hydratase. All of the above steps are optionally repeated one or more times, e.g., to increase the enantioselectivity of the identified nitrilases and nitrile hydratases.

The enantioselective nitrilases or nitrile hydratases produced by the above method optionally comprise an R-selective nitrilase, an R-selective nitrile hydratase, an S-selective nitrilase, or an S-selective nitrile hydratase. Alternatively, the enzymes comprises R-specific nitrilases or nitrile hydratases, or S-specific nitrilases or nitrile hydratases. R- and/or S-selective or specific racemases, esterases, amidases, and the like are also optionally produced according to the methods described herein.

Screening for enantioselectivity typically comprises contacting a racemic mixture of a compound, e.g., a nitrile such as an amino-nitrile, with artificially evolved enantioselective nitrilases or nitrile hydratases encoded by the library of recombinant DNA segments. Such contact typically results in the production of one or more product, e.g., an amide or a carboxylic acid, e.g., in mixtures comprising R and S enantiomers. For enantioselective and enantiospecific enzymes, the amount one enantiomer will be greater than its opposite enantiomer, e.g., an enantiomeric excess will exist of one enantiomer over the other.

The products are separated, e.g., the enantiomers are separated and the product is separated from unreacted substrate. Separation is typically accomplished using, e.g., capillary electrophoresis, chiral capillary electrophoresis or HPLC. Commercial systems for assaying chirality are available and well established. In addition, HPLC systems utilizing chiral columns are also commercially available. Alternatively, nuclear magnetic resonance spectroscopy (NMR) is used to identify any unreacted product and each of the enantiomers in the reaction mixture.

The amount of each enantiomer, e.g., amide or carboxylic acid enantiomer, is then determined and the enantiomeric excess determined according to the following formula: (A−B)/(A+B)×100%. For example, the percentage of R-amide in a mixture of R and S amides is determined or the percentage of an S-carboxylic acid is determined. One or more artificially evolved enantioselective nitrilase is typically identified that produces an excess of about 60% to about 90% or more of the R-carboxylic acid, relative to the S-carboxylic acid, or the S-carboxylic acid relative to the R-carboxylic acid. In preferred embodiments, the artificially evolved enantioselective nitrilase produce about 95% or more, about 99% or more, or about 99.5% or more of the desired amide or carboxylic acid enantiomer, e.g., the R-carboxylic acid or the S-carboxylic acid.

Recombinant enzymes, e.g., nitrilases, nitrile hydratases, and esterases, produced by the methods described herein are also embodiments of the present invention as well as compositions and reaction mixtures comprising the artificially evolved enzymes.

A variety of diversity generating protocols, e.g., for generating enantioselective nitrilases and nitrile hydratases, are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

Descriptions of a variety of diversity generating procedures for generating modified nucleic acid sequences, e.g., as applied to the present invention, R-selective nitrilases or nitrile hydratases, are found in the following publications and the references cited therein: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION”, by del Cardayre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

In brief, several different general classes of sequence modification methods, such as mutation, recombination, etc. are applicable to the present invention and set forth, e.g., in the references above. That is, enantioselective or enantiospecific nitrilases, nitrile hydratases, esterases, racemases, and/or hydralases are optionally produced by sequence modification, e.g., of various known nitrilase or nitrile hydratases, e.g., those found in Genbank. For example, mutation or recombination of nucleic acids corresponding to the following Genbank accession numbers is optionally used to produce enantioselective nitrilases, e.g., R-selective nitrilases: D12583, D67026, L32589, D13419, E01313, and AB028892. Mutation or recombination of nucleic acids corresponding to the following Genbank accession numbers is optionally used to produce enantioselective nitrile hydratases, e.g., R-selective nitrile hydratases: M60264, X64359, E03179, X64360, D14454, M74531, AF257489, E08304, D90216, and E13931.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants are described in several of the references above, e.g., in Stemmer (1994) Proc. Natl. Acad. Sci. USA 91:10747-10751. Thus nitrile hydratases, e.g., from Pseudomonas putida, are optionally digested with DNAse and then ligated or reassembled using PCR to create an R-selective nitrile hydratase.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Thus two naturally occurring hydratases are optionally recombined in vivo to produce a hydratase with improved enantioselectivity or different enantioselectivity.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al. , filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/ gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01 138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579). Extensive details regarding in silico recombination methods are found in these applications. This methodology is generally applicable to the present invention in providing for recombination of known hydratase or nitrilase sequences in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “SINGLE-STRANDED NUCLEIC ACID TEMPLATE-MEDIATED RECOMBINATION AND NUCLEIC ACID FRAGMENT ISOLATION” by Affholter, U.S. Ser. No. 09/656,549, filed Sep. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity. For example new enantioselective nitrilases are optionally produced as well as nitrile hydratase with increased enantioselectivity. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11: 1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/ or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optirnizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

The result of any of the diversity generating procedures described herein can be the generation of one or more nucleic acids, e.g., a library of diversified nucleic acids encoding artificially evolved nitrilases or nitrile hydratases, which can be selected or screened for nucleic acids that encode proteins with or which confer desirable properties. Following diversification by one or more of the methods herein, or otherwise available to one of skill, any nucleic acids that are produced can be selected for a desired activity or property, e.g. encoding an enantioselective or enantiospecific protein, e.g., one that provides enantioselective or enantiospecific conversion of nitrites to amides or carboxylic acids. This can include identifying any activity that can be detected, for example, in an automated or automatable format, by any of the assays in the art.

For example, in the present invention, bacteria having either natural or artificially-evolved nitrilase or nitrile hydratase activity are identified, e.g., using whole cell assays. In one embodiment, an assay for ammonia is used to detect nitrilase activity. Ammonia is liberated from the nitrile when the nitrile is converted to a carboxylic acid. By detecting the ammonia, cells that contain a protein or peptide having nitrilase activity are identified. The enantioselectivity of the nitrilases or nitrile hydratases are then determined as described herein, e.g., in a further screen.

For example, once a cell or cell colony is identified as having as nitrilase or nitrile hydratase activity, the cells are incubated with a racemic mixture of the desired substrate and centrifuged. The resulting supernatant is used to screen for enantioselective activity. The samples are typically derivatized using fluorescein and separated, e.g., using chiral capillary electrophoresis as described in Example 3. The enantiomeric excess is then optionally determined, e.g., to identify one or more enantioselective enzyme or nucleic acid encoding an enantioselective enzyme. Prior to screening for activity and enantioselectivity, libraries of artificially evolved enzymes, or cells transformed with polynucleotides encoding such enzymes, are optionally prescreened, e.g., for proper protein folding using the methods described, e.g., in Waldo et al. Nature Biotechnology 17,:691 (July 1999); and WO 99/31266.

Alternatively, the screening for activity and enantioselectivity is performed in a single screen, e.g., by mixing evolved proteins, e.g., isolated or in whole cells, with a racemic nitrile mixture and performing capillary electrophoresis to identify products and determine the chirality of each product. In other embodiments, a cell supernantant is analyzed by NMR to determine how much of each enantiomer is produced, e.g., how much R-amide is produced from a nitrile hydratase reaction, e.g., in comparison to the S-amide.

For example, evolved enzymes are typically screened by contacting a racemic mixture of nitrites with the proteins encoded by the nucleic acids to be screened. The resulting products are separated and identified, e.g., using chiral capillary electrophoresis as described herein. The percentage of each enantiomer produced is determined and those nucleic acids that encode enantioselective enzymes or enantiospecific enzymes are identified.

Chiral capillary electrophoresis is optionally performed in a high throughput manner, e.g., as described in an example below. Alternatively, commercial systems are available for assaying chirality. In addition, the best improved candidate nitrilases and/or nitrile hydratases can be analyzed thoroughly by HPLC, e.g., using chiral columns that are commercially available. Alternate methods of screening include chiral gas chromatography (GC), GC/mass spectrometry, NMR spectroscopy, magnetic resonance imaging, HTP Maldi mass spectrometry screening for measuring chirality; derivatization followed by separation via chromatography, e.g., gas chromatography, gel filtration chromatography, and the like, ion exchange, and the like, followed by optical rotation measurements; HTP flow through optical rotation measurements using e.g., PDR chiral detection systems (PDR Chiral, Inc.). For other detection and screening systems, see, e.g., “Reaction Microarrays; determination of ee in HTP on a microarray format,” Korbel et al. J. Am. Chem. Soc., 2001, 123, 361-362; and “Enantiomeric analysis of pharmaceutical compounds by ion/molecule reactions,” by Grigorean and Lebrilla, Anal. Chem. Apr. 15, 2001; 73(8):1684-91. Further methods include, but are not limited to, reacting the product of the reaction with a chiral derivatizing agent such as a mosher's ester (couple to amino groups). The enantiomeric excess can then be directly measured by NMR. In addition, a large number of methods exist to chemically derivatize the products of a nitrilase reaction, e.g., with a chirally pure reagent. This forms a diastereomeric pair of derivatized products that are then readily separated and resolved/quantified, e.g., by tlc, ce, gc, ms, and the like. In addition, a variety of related (or even unrelated) properties can be evaluated, in serial or in parallel, at the discretion of the practitioner.

In other embodiments, cells comprising nucleic acids encoding candidate nitrilases, nitrile hydratases, racemases, amidases, esterases, or the like, are incubated with a racemic mixture of a desired substrate and the results are analyzed by mass spectrometry, e.g., high throughput mass spectrometry as described in published PCT application, WO 00/48004, High Throughput Mass Spectrometry, by Raillard et al., published Aug. 17, 2000. For example, cells transformed with a library of recombinant DNA segments encoding putative enantioselective nitrilases are optionally incubated with a nitrile for a desired time period, e.g., about 10 minutes to about 12 hours at temperatures ranging from about 4° C. to about 70° C. The cell supernatant is then analyzed by mass spectrometry for the presence of the carboxylic acid due to nitrilase activity on the nitrile. Those cells comprising a nucleic acid encoding a functional nitrilase are then optionally incubated with a racemic mixture of the desired nitrile, and analyzed, e.g., by capillary electrophoresis, to determine whether the nitrilases are enantioselective. Examples of the above screening procedures are provided below.

VI. Examples

### EXAMPLE 1

### Optimization of Nitrilase Genes by DNA Shuffling

Two homologous nitrilase genes, with sequences represented by SEQ ID NO: 1 and SEQ ID NO: 3, are used as substrates for DNA shuffling. The nitrilase genes are fragmented and the fragments reassembled together, as described, for example, in WO 97/20078, to form a library of chimeric nitrilase variants. After DNA shuffling, the protein coding regions are amplified from the shuffling reaction by PCR, e.g., using pools of primers corresponding to the 5′ and 3′ ends of the nitrilase genes. The forward primers are based on the sequence at the translational start site of each of the nitrilase genes. The sequences are modified to include an SfiI site to facilitate cloning into an expression vector. The reverse primers are based on the sequence around the translation termination site of the nitrilase genes. These primers include a coding sequence for six consecutive histidine residues and an SfiI restriction site at the end of the nitrilase protein-coding region. A His tag is optionally used later, e.g., to purify the proteins produced by E. coli cells that contain the shuffled genes. The amplified PCR products are digested with SfiI restriction endonuclease and cloned into an expression vector, which is then used to transform E. coli cells.

### EXAMPLE 2

### Detection of Nitrilase-producing Bacteria

To identify bacteria producing either natural or artificially-evolved nitrilase activity, a whole-cell assay was developed. Because the action of a nitrilase on a nitrile results in the liberation of ammonia in an amount equimolar to the desired carboxylic acid product, a generic assay for quantifying ammonia is optionally used for the detection of nitrilase activity on a wide range of nitrile substrates. One assay used here was a modification of a commercially-available system for the detection of urea in blood or urine, e.g., a colorimetric, liquid assay based on the method of Fawcett and Scott, (A rapid and precise method for the determination of urea, J. Clin Path, (1960) 13: 156-159). In the example assay, the liberated ammonia reacts with sodium phenate and hypochlorite to produce indophenol, which has a blue color and an absorption maximum around 630 nm.

For rapid identification of nitrilase-producing bacteria, suspensions of the bacteria in buffer (typically phosphate buffer, pH 5-10) are incubated with the desired nitrile substrate, e.g., at a concentration of at least about 10 mM, for a desired period of time ranging from about 10 minutes to overnight at temperatures between about 4° C. and about 70° C. After incubation, the culture supernatant is assayed for the presence of ammonia, with a blue color indicating functional nitrilase activity. Typically, 10 μl of culture supernatant is mixed with 20 μl of phenol-nitroprusside reagent (Sigma) to which 20 μl of alkaline hypochlorite solution (Sigma) is then added. The reaction is allowed to develop at room temperature for about 4 minutes to about 1 hour, and then the absorbance at 570 nm is measured spectrophotometrically. For quantitative determination of nitrilase activity, an ammonia standard curve is used, to which suitable dilutions of the culture supernatant in water are compared.

For nitrile compounds that release ammonia when they decompose, an alternate screening protocol is typically used. For example, amino nitriles are a class of nitrites that often decompose in this manner. For the specific example of phenylglycine nitrile, a high throughput mass spectrometry assay was developed for the detection of phenylglycine. See, e.g., WO 00/48004, High Throughput Mass Spectrometry, by Raillard et al., published Aug. 17, 2000. In this case, the cells are resuspended in morpholine buffer rather than phosphate buffer. After incubation with phenylglycine nitrile for the desired period of time, the cells are removed by centrifugation and the culture supernatant is further clarified by filtration before injection into the mass spectrometer. Quantitation is typically by comparison to a known standard.

In the manner described above, cells producing nitrilases, e.g., functional nitrilases that convert a nitrile to a carboxylic acid are identified. Enantioselectivity of the various nitrilases identified is determined as described below.

### EXAMPLE 3

### Screening Nitrilase Variants for Enantioselectivity

E. coli transformants containing nitrilases that were active on phenylglycine nitrile as determined by mass spectrometry analysis are further screened for enantioselectivity, typically using capillary electrophoresis. Nitrilase-expressing transformants are incubated with a racemic mixture of D- and L-phenylglycine nitrile for a desired period of time and screened as described below.

Sample Prep: Whole cell samples are centrifuged for 10 minutes at 4000 rpm. 20 μl of supernatant is drawn off and plated into 96 well plates.

Fluorescein Derivitization: 40 μl of 1.25 mM FITC (fluorescein isothiocyanate) dissolved in DMSO is added to each sample (2:1). Samples are incubated in the dark at room temperature for at least about 30 minutes.

Sample Dilution: Derivitized samples are diluted 1:400 in ddH20 (18 ohm).

Separation Protocol: Separations are done using the method described in Reetz et al., Angew. Chem. Int. Ed. 2000, 39(21) 3891-3893, using a SpectruMedix HTS 9610 machine, with an array of 96, 50 μM I.D capillaries of approximately 35 cM in length each and a running buffer (40 mM TetraBorate-HEPES pH 7.5, 30 mM Gamma-Cyclodextrin, and 20% IsoPropyl Alcohol). The sample is vacuum injected at 1 psi and the system is then set for electrophoresis at 15 kV for 40 minutes.

### EXAMPLE 4

### Mass Spectrometry (MS) Screening Process

One sample, e.g., 5 μl, was drawn from a 96-well microtiter plate at a speed of about one sample about every 45 seconds and was injected into the mass spectrometer (Micromass Quattro LC, triple quadrupole mass spectrometer) without any separation. The sample was carried into mass spectrometer by a mobile phase (50/50 water/methanol) at a flow rate of about 300 uL/min.

Each injected sample was ionized by a negative electrospray ionization process (needle voltage is about −3.0 KV, cone voltage about 30 V, source temperature about 125° C., desolvation temperation about 250° C., cone gas flow about 90 L/Hr and desolvation gas flow about 600 L/Hr), and the molecular ions (m/z 150) formed during this process were selected by first quadrupole for collision induced dissociation (CID) process in the second quadrupole, where the pressure was set at about 5×10⁻⁴ mBar and the collision energy was adjusted to about 7 eV. The third quadrupole was set for only allowing one of the daughter ions (m/z 106) produced from the parent ions (m/z 150) to get into the detector for signal recording. Both quadrupoles (first and third) were set at unit resolution while the photomultiplier was operated at 650 V.

### EXAMPLE 5

### One Pot Synthesis of Chiral Amino Acids or Hydroxyl Acids

Amino acids and the corresponding hydroxyl acids are optionally synthesized in a one pot procedure using an enantioselective nitrilase, e.g., produced as described above. Starting materials are optionally ketones or aldehydes as shown below. The ketones and/or aldehydes are converted to amino nitrites or hydroxyl nitrites, which are then converted to the corresponding amino acid or hydroxyl acid, e.g., using a nitrilase of the invention, as shown below. The formation of an amino nitrile or cynanohydrin from the corresponding aldehyde or ketone is a reversible process. This allows the production of a chiral hydroxyl or amino acid using a stereoselective nitrilase, e.g., with a yield of about 60% or more, about 90% or more, about 95% or more, or about 99% or more.

### One Pot Synthesis of Chiral Hydroxyls and Amino Acids

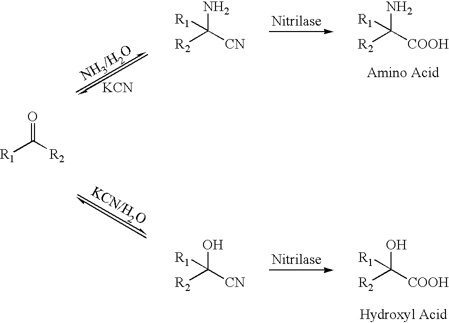

R₁ and R₂ are typically H, alkyl, alkyenyl, alkynyl, aryl, heteroaryl, cycloalkyl, heterocyclic, or the like.

Typical reaction procedure: To a reaction vessel with 1M KCN and 1 M NH₄Cl in 100 mM phosphate pH 7 buffer stirring at room temperature, a solution of 1M aldehyde in methanol is slowly added. After about 2 hr of stirring, nitrilase enzyme (using either whole cells or purified enzyme) is added and incubated by shaking at 37° C. for about 2 hrs. The desired product is monitored by HPLC and recovered by crystallization. [For wild type Nitrilase (1 mg/ml), for whole cells (8 mg/ml)] Smaller scale reactions, e.g., with volume<500 μL, typically do not need shaking, but are simply incubated, e.g., in the reaction vessel.

Estimated reaction conditions vary depending on the aldehyde or ketone used as a starting material. Typically the temperature range varies from about 4° C. to about 70° C., with an estimated pH range from about 5 to about 8. Reaction time typically varies from about 4 to about 12 hours, with yields that range from about 60% to about 100%. For 1M substrate concentration, the purified enzyme concentration is typically about 1-100 mg/mL. For whole cells the concentration is typically about: 10-800 mg/mL. Typical substrate concentrations are at least about 10 mM, more preferably at least about 100 mM or more or in some cases, 1.3 M or greater. In some embodiments, the substrate concentration is greater than about 1M or greater than about 1.3 M.

Because many aldehydes are not very soluble in aqueous solvent [example: benzaldehyde (80 mM)], the use of methanol is optionally used to increase the solubility of benzaldehydes to the molar scale. However, in some embodiments the nitrilase enzyme is immobilized and a continuous flow reaction system is used, e.g., in aqueous solvent at a mM scale.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of converting a nitrile to an amide, the method comprising: contacting the nitrile with an artificially evolved enantioselective nitrile hydratase, thereby forming the amide.

2. The method of claim 1, wherein the nitrile comprises a racemic mixture.

3. The method of claim 1, wherein the nitrile comprises an amino nitrile.

4. The method of claim 1, wherein the amide is an R-amide.

5. The method of claim 1, wherein the enantioselective nitrile hydratase comprises an R-selective nitrile hydratase or an S-selective nitrile hydratase.

6. The method of claim 1, wherein the enantioselective nitrile hydratase comprises an R-selective nitrile hydratase and the nitrile comprises a first racemic mixture.

7. The method of claim 6, wherein contacting the first racemic mixture with the R-selective nitrile hydratase results in an R-amide and an unconverted S-nitrile, the method further comprising:
racemizing the unconverted S-nitrile to produce a second racemic mixture; and,
contacting the second racemic mixture with the R-selective nitrile hydratase.

8. The method of claim 1, wherein the artificially evolved enantioselective nitrile hydratase is produced by recombining two or more nucleic acids encoding a nitrile hydratase.

9. The method of claim 8, wherein recombining the two or more nucleic acids comprises recombining two or more nucleic acids corresponding to the following Genbank accession numbers: M60264, X64359, E03179, X64360, D14454, M74531, AF257489, E08304, D90216, and E13931.

10. The method of claim 1, wherein the enantioselective nitrile hydratase is produced by mutating one or more nitrile hydratase.

11. The method of claim 10, wherein mutating the one or more nitrile hydratase comprises mutating one or more nucleic acid corresponding to the following Genbank accession numbers: M60264, X64359, E03179, X64360, D14454, M74531, AF257489, E08304, D90216, and E13931.

12. The method of claim 1, wherein the enantioselective nitrile hydratase is produced by error prone PCR or assembly PCR.

13. A method of converting a nitrile to a carboxylic acid, the method comprising: contacting the nitrile with an artificially evolved enantioselective nitrilase, thereby forming the carboxylic acid.

14. The method of claim 13, wherein the nitrite comprises a racemic mixture.

15. The method of claim 13, wherein the nitrile comprises an amino nitrile.

16. The method of claim 13, wherein the carboxylic acid comprises an R-carboxylic acid or an S-carboxylic acid.

17. The method of claim 13, wherein the nitrite comprises an amino nitrite and the carboxylic acid comprises an amino acid.

18. The method of claim 17, wherein the amino nitrite comprises a racemic mixture and the amino acid comprises an optically active amino acid.

19. The method of claim 18, wherein the amino acid comprises an R-amino acid or an S-amino acid.

20. The method of claim 13, wherein the enantioselective nitrilase comprises an R-selective nitrilase or an S-selective nitrilase.

21. The method of claim 13, wherein the enantioselective nitrilase comprises an R-selective nitrilase and the nitrile comprises a first racemic mixture.

22. The method of claim 21, wherein contacting the first racemic mixture with the R-selective nitrilase results in an R-carboxylic acid and an unconverted S-nitrile, the method further comprising:
racemizing the unconverted S-nitrile to produce a second racemic mixture; and,
contacting the second racemic mixture with the R-selective nitrilase.

23. The method of claim 13, wherein the artificially evolved enantioselective nitrilase is produced by recombining two or more nucleic acids encoding a nitrilase.

24. The method of claim 23, wherein recombining the two or more nucleic acids comprises recombining two or more nucleic acids corresponding to the following Genbank accession numbers: D12583, D67026, L32589, D13419, E01313, and AB028892.

25. The method of claim 13, wherein the artificially evolved enantioselective nitrilase is produced by recombining three or more homologous nucleic acids, wherein each of the three or more homologous nucleic acids is derived from a parental nucleic acid encoding a nitrilase.

26. The method of claim 25, wherein recombining the three or more homologous nucleic acids comprises recombining three or more nucleic acids derived from one or more nucleic acid corresponding to the following Genbank accession numbers: D12583, D67026, L32589, D13419, E01313, and AB028892.

27. The method of claim 13, wherein the enantioselective nitrilase is produced by mutating one or more nitrilase.

28. The method of claim 27, wherein mutating the one or more nitrilase comprises mutating one or more nucleic acid corresponding to the following Genbank accession numbers: D12583, D67026, L32589, D13419, E01313, and AB028892.

29. The method of claim 27, comprising mutating the one or more nitrilase by site directed mutagenesis, cassette mutagenesis, random mutagenesis, recursive ensemble mutagenesis, or in vivo mutagenesis.

30. The method of claim 13, wherein the enantioselective nitrilase is produced by error prone PCR or assembly PCR.

31. A method of making an amino acid, the method comprising:
(i) contacting an amino nitrile with an artificially evolved enantioselective nitrile hydratase, thereby producing an amide; and,
(ii) contacting the amide with an amidase, thereby making the amino acid.

32. The method of claim 31, wherein the enantioselective nitrile hydratase comprises an R-selective nitrile hydratase or an S-selective nitrile hydratase.

33. The method of claim 31, wherein the artificially evolved enantioselective nitrile hydratase is produced by recombining two or more nucleic acids encoding a nitrile hydratase.

34. The method of claim 33, wherein recombining the two or more nucleic acids comprises recombining two or more nucleic acids corresponding to the following Genbank accession numbers: M60264, X64359, E03179, X64360, D14454, M74531, AF257489, E08304, D90216, and E13931.

35. The method of claim 31, wherein the artificially evolved enantioselective nitrile hydratase is produced by recombining three or more homologous nucleic acids, wherein each of the three or more homologous nucleic acids is derived from a parental nucleic acid encoding a nitrile hydratase.

36. The method of claim 35, wherein recombining the three or more homologous nucleic acids comprises recombining three or more nucleic acids derived from one or more nucleic acid corresponding to the following Genbank accession numbers: M60264, X64359, E03179, X64360, D14454, M74531, AF257489, E08304, D90216, and E13931.

37. The method of claim 31, wherein the enantioselective nitrile hydratase is produced by mutating one or more nitrile hydratase.

38. The method of claim 37, wherein mutating the one or more nitrile hydratase comprises mutating one or more nucleic acid corresponding to the following Genbank accession numbers: M60264, X64359, E03179, X64360, D14454, M74531, AF257489, E08304, D90216, and E13931.

39. The method of claim 37, comprising mutating the one or more nitrile hydratase by site directed mutagenesis, cassette mutagenesis, random mutagenesis, recursive ensemble mutagenesis, or in vivo mutagenesis.

40. The method of claim 31, wherein the enantioselective nitrile hydratase is produced by error prone PCR or assembly PCR.

41. The method of claim 31, wherein the amino nitrile comprises a first racemic mixture.

42. The method of claim 41, step (i) resulting in an R-amide and an unconverted S-amino nitrile, the method further comprising:
(iii) racemizing the S-amino nitrile, resulting in a second racemic mixture; and,
(iv) contacting the second racemic mixture with the enantioselective nitrile hydratase.

43. The method of claim 31, wherein the amide comprises an R-amide.

44. The method of claim 31, wherein the amidase comprises a non-selective amidase.

45. A reaction mixture comprising an amino nitrile and an R-selective nitrile hydratase, an R-selective nitrilase, an S-selective nitrile hydratase, or an S-selective nitrililase.

46. The reaction mixture of claim 45, wherein R-selective nitrile hydratase, the R-selective nitrilase, the S-selective nitrile hydratase, or the S-selective nitrilase comprises an artificially evolved nitrilase or an artificially evolved nitrile hydratase.

47. The reaction mixture of claim 45, wherein the artificially evolved R-selective nitrile hydratase, R-selective nitrilase, S-selective nitrile hydratase, or S-selective nitrililase is produced by recombining two or more nucleic acids encoding a nitrile hydratase or a nitrilase.

48. The reaction mixture of claim 47, wherein recombining the two or more nucleic acids comprises recombining two or more nucleic acids corresponding to the following Genbank accession numbers: M60264, X64359, E03179, X64360, D14454, M74531, AF257489, E08304, D90216, and E13931.

49. The reaction mixture of claim 47, wherein recombining the two or more nucleic acids comprises recombining two or more nucleic acids corresponding to the following Genbank accession numbers: D12583, D67026, L32589, D13419, E01313, and AB028892.

50. The reaction mixture of claim 45, wherein the R-selective nitrile hydratase, the R-selective nitrilase, the S-selective nitrile hydratase, or the S-selective nitrilase is produced by mutating one or more nitrile hydratase or nitrilase.

51. The reaction mixture of claim 50, wherein mutating the one or more nitrile hydratase comprises mutating one or more nucleic acid corresponding to the following Genbank accession numbers: M60264, X64359, E03179, X64360, D14454, M74531, AF257489, E08304, D90216, and E13931.

52. The reaction mixture of claim 50, wherein mutating the one or more nitrilase comprises mutating one or more nucleic acid corresponding to the following Genbank accession numbers: D12583, D67026, L32589, D13419, E01313, and AB028892.

53. The reaction mixture of claim 50, comprising mutating the one or more nitrile hydratase or nitrilase by site directed mutagenesis, cassette mutagenesis, random mutagenesis, recursive ensemble mutagenesis, or in vivo mutagenesis.

54. The reaction mixture of claim 45, wherein the R-selective nitrile hydratase, the R-selective nitrilase, the S-selective nitrile hydratase, or the S-selective nitrilase is produced by error prone PCR or assembly PCR.

55. The reaction mixture of claim 45, wherein the reaction mixture comprises the R-selective nitrile hydratase or the S-selective nitrile hydratase and an amidase.

56. The reaction mixture of claim 55, wherein the amidase comprises a non-enantioselective amidase.

57. The reaction mixture of claim 45, wherein the amino nitrile comprises a racemic mixture.

58. The reaction mixture of claim 45, wherein the reaction mixture further comprises an R-amino acid.

59. The reaction mixture of claim 45, wherein the reaction mixture further comprises an amide.

60. The reaction mixture of claim 59, wherein the amide comprises an R-amide.

61. A method of producing a nucleic acid encoding an enantioselective nitrilase or an enantioselective nitrile hydratase, the method comprising:
(i) providing a population of DNA fragments, which DNA fragments collectively encode at least one parental nitrilase or nitrile hydratase;
(ii) recombining the DNA fragments to produce a library of recombinant DNA segments;
(iii) optionally repeating steps (i) and (ii);
(iv) screening the library of recombinant DNA segments to identify at least one recombinant DNA segment that encodes an artificially evolved enantioselective nitrilase or enantioselective nitrile hydratase; and,
(v) optionally repeating steps (i) through (iv) one or more times.

62. The method of claim 61, wherein the one or more parental nitrilase comprises one or more nitrilase corresponding to one or more of the following Genbank accession numbers: D12583, D67026, L32589, D13419, E01313, and AB028892.

63. The method of claim 61, wherein the one or more parental nitrile hydratase comprises one or more nitrile hydratase corresponding to one or more of the following Genbank accession numbers: M60264, X64359, E03179, X64360, D14454, M74531, AF257489, E08304, D90216, and E13931.

64. The method of claim 61, wherein the enantioselective nitrilase or nitrile hydratase comprises an R-selective nitrilase, an R-selective nitrile hydratase, an S-selective nitrilase, or an S-selective nitrile hydratase.

65. The method of claim 61, wherein screening comprises
(a) contacting a racemic mixture of a nitrile with the artificially evolved enantioselective nitrilase, thereby producing one or more carboxylic acids; and,
(b) determining a percentage of the one or more carboxylic acids comprising an R-carboxylic acid and a percentage of the one or more carboxylic acids comprising an S-carboxylic acid; and,
(c) identifying one or more artificially evolved enantioselective nitrilase that produced about 90% or more of the R-carboxylic acid or the S-carboxylic acid.

66. The method of claim 65, step (b) further comprising separating the one or more carboxylic acids by HPLC.

67. The method of claim 65, step (b) further comprising performing nuclear magnetic resonance spectrometry on the one or more carboxylic acids.

68. The method of claim 65, comprising identifying one or more artificially evolved enantioselective nitrilase producing about 95% or more, about 99% or more, or about 99.5% or more of the R-carboxylic acid or the S-carboxylic acid.

69. The method of claim 61, wherein screening comprises
(a) contacting a racemic mixture of a nitrile with the artificially evolved enantioselective nitrile hydratase, thereby producing one or more amides; and,
(b) determining a percentage of the one or more amides comprising an R-amide and a percentage of the one or more amides of amides comprising an S-amide;
(c) identifying one or more artificially evolved enantioselective nitrile hydratase producing about 90% or more of the R-amide or the S-amide.

70. The method of claim 69, step (b) further comprising separating the one or more amides by HPLC.

71. The method of claim 69, step (b) further comprising performing nuclear magnetic resonance spectroscopy on the one or more amides.

72. The method of claim 69, comprising identifying one or more artificially evolved enantioselective nitrile hydratase producing about 95% or more, about 99% or more, or about 99.5% or more of the R-amide or the S-amide.

73. The method of claim 61, wherein screening comprises:
(a) transforming one or more cell with the library of recombinant DNA segments;
(b) contacting the one or more cell with a nitrile, thereby producing one or more carboxylic acid; and,
(c) detecting one or more carboxylic acid, thereby identifying one or more member of the library of recombinant DNA segments, which one or more member encodes a nitrilase polypeptide;
(d) contacting the one or more member of the library of recombinant DNA segments with a racemic mixture of the nitrile, resulting in one or more products;
(e) separating the one or more products into a first enantiomer and a second enantiomer;
(f) determining an enantiomeric excess of either the first enantiomer or the second enantiomer, thereby identifying one or more nucleic acid encoding an enantioselective nitrilase.

74. The method of claim 73, step (c) comprising detecting the one or more carboxylic acid by detecting ammonia, which ammonia is liberated when the nitrilase polypeptide converts the nitrile to the carboxylic acid.

75. The method of claim 73, step (c) comprising detecting the one or more carboxylic acid by mass spectrometry.

76. The method of claim 73, step (f) comprising determining a percentage of the first enantiomer in the one or more products and a percentage of the second enantiomer in the one or more products.

77. A recombinant nitrilase or nitrile hydratase produced by the method of claim 61.

78. A method of converting a first enantiomer of a target molecule to a second enantiomer of the target molecule, the method comprising:
(a) converting the first enantiomer of the target molecule to an activated target molecule, the activated target molecule comprising a first enantiomer of the activated target molecule or a racemic mixture comprising the first enantiomer of the activated target molecule and a second enantiomer of the activated target molecule;
(b) contacting the activated target molecule with a racemase and an enantioselective enzyme, wherein
(i) the racemase continuously converts the first enantiomer of the activated target molecule to a racemic mixture comprising the first enantiomer of the activated target molecule and the second enantiomer of the activated target molecule; and
(ii) the enantioselective enzyme converts the second enantiomer of the activated target molecule to the second enantiomer of the target molecule.

79. The method of claim 78, wherein the target molecule comprises an amino acid, a carboxylic acid, an ester, an amine, or an alcohol.

80. The method of claim 78, wherein the activated target molecule comprises a hydrolyzed target molecule.

81. The method of claim 78, wherein the activated target molecule comprises an ester.

82. The method of claim 78, wherein the first enantiomer of the target molecule comprises an L-amino acid and the second enantiomer of the target molecule comprises a D-amino acid.

83. The method of claim 78, wherein the racemase comprises an artificially evolved racemase.

84. The method of claim 78, wherein the racemase and the enantioselective enzyme comprise a fusion enzyme.

85. The method of claim 78, wherein the enantioselective enzyme comprises an esterase or an amidase.

86. The method of claim 78, wherein the enantioselective enzyme comprises an artificially evolved enzyme.

87. The method of claim 78, step (b) continuing until substantially all of the first enantiomer of the target molecule is converted into the second enantiomer of the target molecule.

88. The method of claim 87, wherein about 90% or more of the first enantiomer of the target molecule is converted into the second enantiomer of the target molecule.

89. The method of claim 87, wherein about 95% or more of the first enantiomer of the target molecule is converted into the second enantiomer of the target molecule.

90. A method of making an amino acid, the method comprising:
(a) converting an aldehyde or ketone to an amino nitrile;
(b) contacting the amino nitrile with an enantioselective nitrilase, which nitrilase enantioselectively converts the amino nitrile to an amino acid.

91. The method of claim 90, wherein step (a) and step (b) are performed in a single reaction.
